(19) United States
(12) Patent Application Publication
(10) Pub. No.: US 2010/0260683 A1
(43) Pub. Date: Oct. 14, 2010

(54) THERMOLABILE LIPOSOME WITH CONTROLLED RELEASE TEMPERATURE

(75) Inventors: Hansjorg EIBL, Bovenden-Eddigehausen (DE); Lars LINDNER, Munchen (DE)

Correspondence Address: ROTHWELL, FIGG, ERNST & MANBECK, P.C., 1425 K STREET, N.W., SUITE 800, WASHINGTON, DC, 20005, US

(73) Assignee: MAX-PLANCK-GESELLSCHAFT ZUR FORDERUNG DER WISSENCHAFTEN E.V., Munchen (DE)

(21) Appl. No.: 12/825,475

(22) Filed: Jun. 29, 2010

Related U.S. Application Data
(62) Division of application No. 10/468,116, filed on Mar. 8, 2004.

(30) Foreign Application Priority Data
Feb. 15, 2001 (DE) 101 07 165.5

(51) Int. Cl. A61B 5/00; A61K 9/127; A61P 43/00

(52) U.S. Cl. 424/9.6; 424/450

(57) ABSTRACT

The invention relates to a thermolabile liposome with controlled release temperature for the liposome content, which is essentially formed by at least one phosphatidyl choline with a main transition temperature ranging from 0 to 80° C. and 2 to 15 percent by weight of phosphatidyl oligoglycerin.

### CROSS REFERENCE TO RELATED APPLICATION

This application is a divisional of U.S. Ser. No. 10/468,116, filed Mar. 8, 2004, which application is a 35 USC §371 National Phase Entry Application from PCT/EP02/01653, filed Feb. 15, 2002, and designating the U.S.

### DESCRIPTION

The invention relates to a thermolabile liposome with controlled release temperature for the liposome contents, in particular a liposome which is stable at 37° C. in serum and with a controlled release temperature of between 40 and 80° C.

Liposomes are artificially formed vesicles consisting of lipid bilayers which enclose an aqueous compartment (Bangham et al. 1965). Originally also utilized as a model system for a cell membrane, liposomes were recently developed further, especially for pharmaceutical transport. Liposomes can in this case increase the tolerability of active compounds (lowering of the active toxicity of amphotericin B due to liposomal formulation (AmBisome®) by the factor 75 (Proffitt et al. 1991)). However, they also open up the possibility of transporting pharmaceuticals into diseased tissue in a controlled manner (Forssen et al. 1992). After intravenous administration, liposomes are mainly taken up in cells of the reticuloendothelial system (RES) of the liver and spleen (Gregoriadis and Nerunhun 1974). In order to be able to utilize liposomes as pharmaceutical vehicles for cells outside the RES, it was attempted to increase the circulation time of the liposomes in the blood. Especially in tumors, which are often very highly vascularized (Jain 1996) and whose vessels are particularly permeable due to dilated interendothelial connections, a large number of fenestrations and discontinuous basal membranes (Murray and Carmichael 1995), the probability of absorption of liposomes would be massively increased thereby.

A first problem in the use of liposomes for the transport of active compounds or labeling substances in body fluids therefore lies in the increase in the circulation time in the serum. It has in fact already been found that by means of covalent binding of methoxypolyethylene glycols to the liposomal membrane the premature recognition of the liposomes by the RES can be prevented and thus the circulation time can be improved. In addition to an improvement in the circulation time, there is, however, great interest in the possibility of achieving, by the action of temperature, a release of the liposome contents in a controlled manner at a specific temperature.

The invention is therefore based on the object of creating a liposome which has a significantly improved half-life in the serum, compared with the customary half-life of known liposomes in the order of magnitude of around 4 hours, and which is created such that the contents of the liposomes are rapidly released at a specific temperature.

This object is achieved according to the invention by means of a liposome with controlled release temperature for the liposome contents, which is characterized in that it is essentially formed from at least one phosphatidylcholine having a main transition temperature which lies in the range from 0 to 80° C. and 2 to 15% by weight of phosphatidyloligoglycerol.

Liposomes synthesized according to the invention essentially have improved half-lives of up to 20 hours in the serum and the ingredient(s) can be released rapidly and completely at a predetermined preselected temperature by suitable choice of the components and amounts of the components depending on their main transition temperature.

Preferably, the liposome according to the invention is composed of 65 to 75% of dipalmitoyllecithin (1,2-di-palmitoylglycero-3-phosphocholine), 10 to 30% of distearoyllecithin (1,2-distearoylglycero-3-phospho-choline) and 5 to 10% of dipalmitoylphosphoglyceroglycerol. This preferred composition is stable at 37° C. in the serum, but releases the contents rapidly on exceeding a temperature of 40° C.

The abovementioned preferred composition of the liposome according to the invention can be made to measure for other temperature ranges by choice of components having the main transition temperature suitable in each case. In table 1, the main transition temperatures (T_{M}) of phosphatidylcholines are indicated whose main transition temperature lies in the range from 0 to 80° C. The main transition temperatures are recognizable as from the table, depending on the chain length and the distribution over the positions 1 and 2 of glycero-3-phosphocholine or 1 and 3 of glycero-2-phosphocholine.

TABLE 1

| T_{M} | Phosphatidylcholine |
|---|---|
| 5° C. | 1-palmitoyl-2-oleoyl- |
| 7° C. | 1-stearoyl-2-oleoyl- |
| 11° C. | 1-palmitoyl-2-lauroyl- |
| 14° C. | 1-behenoyl-2-oleoyl- |
| 17° C. | 1-stearoyl-2-lauroyl- |
| 19° C. | 1,3-dimyristoyl- |
| 23° C. | 1,2-dimyristoyl- |
| 27° C. | 1-palmitoyl-2-myristoyl- |
| 33° C. | 1-stearoyl-2-myristoyl- |
| 37° C. | 1-myristoyl-2-palmitoyl |
| 39° C. | 1,3-dipalmitoyl- |
| 41° C. | 1,2-dipalmitoyl- |
| 42° C. | 1-myristoyl-2-stearoyl- |
| 46° C. | 1-stearoyl-3-myristoyl- |
| 48° C. | 1-stearoyl-2-palmitoyl- |
| 52° C. | 1-palmitoyl-2-stearoyl- |
| 53° C. | 1,3-distearoyl- |
| 56° C. | 1,2-distearoyl- |
| 66° C. | 1,2-diarachinoyl- |
| 75° C. | 1,2-dibehenoyl- |
| 80° C. | 1,2-dilignoceroyl- |

The values listed in table 1 show that by use of fatty acids having an uneven chain length and a suitable distribution over the glycerol parent structure virtually any desired temperature can be set in the range from 0 to 80° C. indicated.

The content of phosphatidyloligoglycerols in the liposome according to the invention is essential for the long circulation time necessary in the serum. The phosphatidyloligoglycerols and their preparation are disclosed in DE 196 22 224. Preferably, dipalmitoylphosphoglyceroglycerol (DPPG2) is used.

The thermolabile liposomes according to the invention are outstandingly suitable for use in various areas, but in particular in the context of regional deep hyperthermia. Regional deep hyperthermia, which is used in specialized clinical centers in combination with systemic chemotherapy, presents itself as an ideal technique for tumor-specific liposomal transport and the subsequent release of a pharmaceutical from the liposomal envelope. Thus hyperthermia on the one hand promotes the extravasation of liposomes from tumor capillaries into the interstitium (Gaber et al. 1996). On the other hand, as a result of the heating a release of the pharmaceutical from special thermosensitive liposomes can be induced (Magin and Niesman 1984). Additionally, there are numerous indications of an increased cytotoxic effect of cytostatics (Hahn et al. 1975) and of an immunomodulation (activation of NK cells; Multhoff et al. 1999) by means of regional deep hyperthermia.

The thermolability of the liposomes according to the invention is caused by the phase transition of the phospholipids within the liposomal membrane. If the phase transition temperature is passed through, a short-term membrane instability and subsequent release of the liposomal contents occur.

In the case of the abovementioned regional hyperthermia, the tumor is specifically overheated regionally so that the temperature increases above the threshold temperature for the release of the liposomal contents. Possible liposomal contents here are in particular active compounds which can be used in oncology such as, for example, cytostatics. However, contrast agents, for example gadolinium, carboxy-fluorescein or the like, can also be released on their own or together with an active compound. By use of contrast agents such as gadolinium, a noninvasive thermometry is made possible, in which the temperature achieved which the released gadolinium measures can be determined by MRC. In this use of the liposomes according to the invention, a hyperthermia apparatus coupled with an MRC apparatus is expediently used.

A further type of use for the liposomes according to the invention is found in ophthalmology. On encapsulation of a fluorescent labeling substance, it can be detected where the desired overheating has actually occurred, for example in a laser treatment, by release of the fluorescent active compound such as, for example, carboxyfluorescein.

Analogously to the possibility of use in the eye illustrated, liposomes according to the invention can therefore be used generally to make temperatures achieved determinable subsequently, e.g. if specific heating temperatures or the like are to be determined.

The liposomes according to the invention essentially consist of the substances indicated above, which are preferably present in pure form. Impurities should be kept as low as possible, in particular a cholesterol content which is as low as possible should be present. Liposomes are preferred which are completely free of cholesterol since cholesterol leads to a blurring of the phase transition temperature and thus to an excessively wide thermal transition range.

The thermolabile liposomes according to the invention are prepared in the customary manner by dissolving the lipids, for example, in chloroform or chloroform/water/isopropanol, stripping off the solvent, expediently in vacuo in a rotary evaporator and temperature controlling the lipids using aqueous solutions of the ingredients to be encapsulated at temperatures which lie above the phase transition temperature. The duration of this temperature treatment is expediently 30 to 60 minutes, but can, however, also be shorter or longer. By freezing-thawing processes repeated several times, for example 2- to 5-fold freezing and thawing again, a homogenization takes place. Finally, the lipid suspension obtained is extruded through a membrane of defined pore size at a temperature above the phase transition temperature in order to achieve the desired liposome size. Suitable membranes are, for example, polycarbonate membranes of defined pore size, such as 100 to 200 nm. Finally, ingredient which is optionally not encapsulated can be separated off, for example by column chromatography or the like.

The following examples illustrate the invention further.

### EXAMPLE 1

a) In the manner described above, liposomes are prepared which are composed of 70% of DPPC, 20% of DSPC and 10% of DPPG2. They contain encapsulated carboxyfluorescein. Free carboxyfluorescein was separated off beforehand by column chromatography using Sephadex G75.

b) Chamber Model:

For the intravital microscopic detection of CF release from thermolabile liposomes in the hyperthermia field, the chamber model (A-Mel-3 melanoma of the Syrian hamster) of the Syrian hamster is suitable. In this model, a transparent, dorsal skin chamber is implanted in a Syrian golden hamster. After implantation of the skin chamber, the implantation of cells of the A-Mel-3 melanoma of the hamster takes place on the subcutaneous tissue situated in the chamber. In the course of a few days, a tumor a number of millimeters in size grows within the skin of the back of the hamster. The microcirculation and the fluorescent enrichment within the tumor can be observed using a modified vital microscope. The animals additionally receive a central venous catheter. With the aid of a heat exchanger situated under the skin chamber, a heating of the tumor to 42° C. can be achieved locally. The tumor temperature can be measured directly with the aid of a temperature probe (Endrich 1988).

In addition to vital microscopy, the process of MRT measurement in the chamber model is also established (Pahernik et al. 1999). MRT images can be recorded here analogously to the microscopy.

Table 2 shows the values obtained in vitro.

Liposome composition:

### DPPG:DSPC:DPPG-G2=7:2:1

Great stability in the presence of serum at 37° C. (CF release after 12 hours<7%)

1. A process for controlled release of liposome contents from a thermolabile liposome, wherein the liposome consists essentially of at least one phosphatidylcholine having a main transition temperature which lies in the range from 0 to 80° C., and 2 to 15 percent by weight of phosphatidyloligoglycerol, and the liposome contains no cholesterol, whereby the controlled release is achieved by a change in temperature.

2. The process as claimed in claim 1, wherein the at least one phosphatidylcholine is selected from the group consisting of 1-palmitoyl-2-oleoylglycero-3-phosphocholine, 1-stearoyl-2-oleoyl-3-phosphocholine, 1-palmitoyl-2-lauroyl-glycero-3-phosphocholine, 1-behenoyl-2-oleoyl-glycero-3-phosphocholine, 1-stearoyl-2-lauroyl-glycero-3-phosphocholine, 1,3-dimyristoylglycero-2-phosphocholine, 1,2-dimyristoylglycero-3-phosphocholine, 1-palmitoyl-2-myristoylglycero-3-phosphocholine, 1-stearoyl-2-myristoylglycero-3-phosphocholine, 1-myristoyl-2-palmitoylglycero-3-phosphocholine, 1,3-dipalmitoylglycero-2-phospho-choline, 1,2-dipalmitoylglycero-3-phosphocholine, 1-myristoyl-2-stearoylglycero-2-phosphocholine, 1-stearoyl-3-myristoylglycero-2-phosphocholine, 1-stearoyl-2-palmitoylglycero-3-phosphocholine, 1-palmitoyl-2-stearoylglycero-3-phosphocholine, 1,3-distearoylglycero-2-phosphocholine, 1,2-di-stearoylglycero-3-phosphocholine, 1,2-di-arachinoylglycero-3-phosphocholine, 1,2-di-behenoylglycero-3-phosphocholine and 1,2-di-lignoceroylglycero-3-phosphocholine.

3. The process as claimed in claim 1, wherein the phosphatidyloligoglycerol is dipalmitoylphosphoglyceroglycerol.

4. The process as claimed in claim 1, wherein the liposome consists essentially of 65 to 75% of dipalmitoyllecithin (DPPC), 15 to 25% of distearoyllecithin (DSPC) and 5 to 10% of dipalmitoylphosphoglyceroglycerol (DPPG2).

5. The process as claimed in claim 1, wherein the liposome contents which are released contain an active compound and/or a labeling substance.
